(19) United States
(12) Patent Application Publication
(10) Pub. No.: US 2012/0313759 A1
(43) Pub. Date: Dec. 13, 2012

(54) Tracking system for persons and/or objects

(75) Inventors: Barry J. Markwitz, Solon, OH (US); Paolo Argentieri, Richmond Heights, OH (US); Brent G. Bowers, Cuyahoga Falls, OH (US); Dean M. Chriss, Euclid, OH (US); Jason D. Doyle, Mantua, OH (US); John E. Hansley, II, Lyndhurst, OH (US); John W. Hoffman, Mentor, OH (US); Nicholas F. Papatonis, Cuyahoga Falls, OH (US); Roger W. Stahl, Mantua, OH (US); Mary T. Upham, Brecksville, OH (US)

(73) Assignee: TimeKeeping Systems, Inc.

(21) Appl. No.: 13/134,534

(22) Filed: Jun. 13, 2011

(51) Int. Cl. H04Q 5/22

(52) U.S. Cl. 340/10.1

(57) ABSTRACT

An RFID-based tracking system that tracks persons and/or objects of interest without the need for triangulation techniques is disclosed. The system tracks persons and/or objects of interest by utilizing RFID antennas having a relatively small field of view and positioned relative to functional areas of a facility and/or within passageways between functional areas of the facility. The persons and/or objects of interest to be tracked are provided with an RFID tag. The present invention provides the system user with the ability to determine whether the persons and/or objects of interest are present within a particular functional area of the facility regardless of whether the presence of the person and/or object of interest is continuously detected by the system.

### TECHNICAL FIELD

The present invention relates, in general, to tracking systems and, more particularly, to tracking systems that are utilized to track persons and/or objects of interest.

### BACKGROUND ART

Systems for tracking persons and/or objects of interest in correctional, healthcare, and other facilities presently exist, but they have serious inherent disadvantages that are difficult and expensive to overcome. Such systems typically require that all persons and/or objects of interest be fitted with RFID tags that must be visible to the system at all times in order to make a determination as to the location of a specific person and/or object of interest within the facility.

The amount of hardware required to cover all areas within a large and complex facility makes such systems extremely expensive to install, and the process to install same may be disruptive to the operation of the facility. When present systems provide continuous visibility of persons and/or objects of interest, the systems often provide the system user with more information than required. For example, a system user may need to know if a person of interest has visited a specific area, such as a clinic, while not needing to know the exact location within the clinic that the person of interest visited.

Present tracking systems typically utilize RFID tags and triangulation techniques involving a minimum of two antennas to determine the location of RFID tags carried by persons and/or objects of interest. These systems have a number of significant inherent problems.

Because the transmissions from RFID tags have a relatively short range, and every possible location within the facility must be within the range of at least two spatially separated antennas, large numbers of antennas and associated electronic hardware and cabling are required, which makes triangulation-based RFID tracking systems very expensive. Further exacerbating this problem is the fact that many of the building materials present in correctional, healthcare, and other facilities can significantly affect the accuracy of triangulating systems. For example, concrete walls are typically opaque to the weak signals transmitted by RFID tags because the walls absorb the energy of the signal. Metal doors and furniture are also opaque to these signals since such doors and furniture reflect, rather than absorb, the signals. At the same time, thick acrylic windows, which are sometimes used to separate secured areas within such facilities, are transparent to RFID signals. Because some triangulating systems rely on signal strength measured at each of at least two spatially separated antennas to determine the distance of the RFID tags from each antenna, the various combinations and positions of absorbing, reflecting and transparent materials throughout the facility can adversely affect the accuracy of these systems. This, in turn, further reduces the useable distance between RFID tags and system antennas, necessitating the use of additional antennas, which again increases the high cost of hardware and installation for such systems. Additionally, present RFID tracking systems often require the use of active RFID tags. Active RFID tags are much more expensive than other types of tags, such as passive or battery assisted passive (BAP) RFID tags. Because every person and/or object of interest to be tracked requires an RFID tag, the cost of the RFID tags over time can significantly increase the cost of the overall system.

In addition to the costs involved, the process of installing the numerous antennas, cabling and associated electronic equipment at locations throughout a facility, particularly a correction facility, is disruptive to the operation of the facility. Even with a complex installation, dead zones in which RFID tags cannot be tracked often remain. When visibility of a person and/or object of interest is not available in response to an inquiry by a system user as to the present location of such person and/or object, these systems may provide no useful information. While the continuous, exact, real-time location tracking provided by a properly functioning triangulation-based RFID tracking system can satisfy the needs of many correctional, healthcare, and other facilities, these systems provide, at great cost, functionality that may extend well beyond most of the required needs. Some of the functionality typically goes completely unused due to existing statutory regulations and procedures. For example, such systems in correctional facilities are capable of providing features such as automated head counts, while laws in many jurisdictions require that head counts have visual confirmation of prisoner identity by a correctional facility staff member. Many present systems also provide escape detection means, but the ease with which RFID tags can be removed by inmates makes such detection somewhat unreliable and correctional facilities already have very reliable escape prevention means in place.

The tracking system features that correctional and other facilities typically require and use are those aimed at proving compliance with statutory standards and with limiting liability. For example, a certain amount of recreation time is mandated for correctional facility inmates in most jurisdictions, and inmate tracking systems can be used to show that inmates spent the mandated amount of time in the facility's recreation area. In the same manner, these systems can show that an inmate was in the clinic area of the facility at a given time to help counter claims that the inmate did not receive medical treatment.

Inmate tracking systems are also used for general inmate management, such as determining which inmates are present in certain housing units, and the like. A review of the most-used features of present tracking systems shows that the systems need not determine the exact location of the inmates, but rather the systems must determine when, and if, the inmates are present in certain areas of the facility. For example, a correctional facility may need to determine when a particular inmate visited the clinic without needing to determine the exact location within the clinic at which the inmate was located during every minute of his or her visit. Similarly, these facilities may need to determine whether the inmate has received mandated recreation time, counseling attorney visits, or whether the inmate has been present in the same room or area with another inmate with whom the inmate is not allowed to interact, and the like.

It should be noted that some correctional facilities utilize a camera-based system to track inmate movement but, like the triangulation-based RFD) tracking systems previously discussed, these systems also require that the inmates be continuously within the field of view of at least one of the cameras of the system. Like triangulation-based RFID tracking systems, when such inmate visibility does not exist, the system can provide no information as to the location of the inmate. Additionally, current camera-based systems can only provide recorded video to document events, and these systems must be constantly and diligently monitored if undesirable events are to be avoided. Such systems cannot provide historical information in report format or automatically provide alerts when inmates who are not allowed to interact are present in the same area.

In view of the foregoing inherent problems associated with the prior art tracking systems, it has become desirable to develop a system for tracking persons and/or objects of interest that does not utilize triangulation techniques while retaining most of the benefits of the triangulating systems.

### SUMMARY OF THE INVENTION

The present invention solves the problems associated with the prior art tracking systems, and other problems, by providing an RFID-based tracking system that does not utilize triangulation techniques. The tracking system of the present invention utilizes RFID antennas that have fields of view and RFID tag interrogation ranges, hereinafter collectively referred to as fields of view, which are relatively small. These antennas are positioned relative to functional areas of a facility and/or passageways between functional areas of the facility. The foregoing arrangement allows the use of a lesser number of antennas, each with a field of view covering only a small area within the facility, and further allows the use of inexpensive battery assisted passive (BAP) and/or passive RFID tags. The present invention provides the system user with the ability to determine if, and when, persons and/or objects of interest are present within particular functional areas or rooms within a facility regardless of whether the presence of the person and/or object of interest can be continuously detected by the system.

### BRIEF DESCRIPTION OF THE DRAWINGS

FIG. 1 is a schematic diagram of the fundamental components utilized by the tracking system of the present invention.

FIG. 2 is a diagram of the possible states of an RFID tag and illustrates how the states are determined by the software utilized by the tracking system of the present invention.

FIG. 3 is an illustration of an installation of the preferred embodiment of the present invention in a simplified setting.

FIG. 4 is an illustration of antenna placement in the installation of the preferred embodiment of the present invention shown in FIG. 3.

FIG. 5 is an illustration of another antenna placement in the installation of the preferred embodiment of the present invention shown in FIG. 3.

FIG. 6 is an illustration of antennas positioned in a back-to-back relationship and shows the overlapping fields of view of the antennas.

FIG. 7 is an illustration of the field of view of a single antenna to cover areas on opposite sides of a wall.

### DESCRIPTION OF THE PREFERRED EMBODIMENT

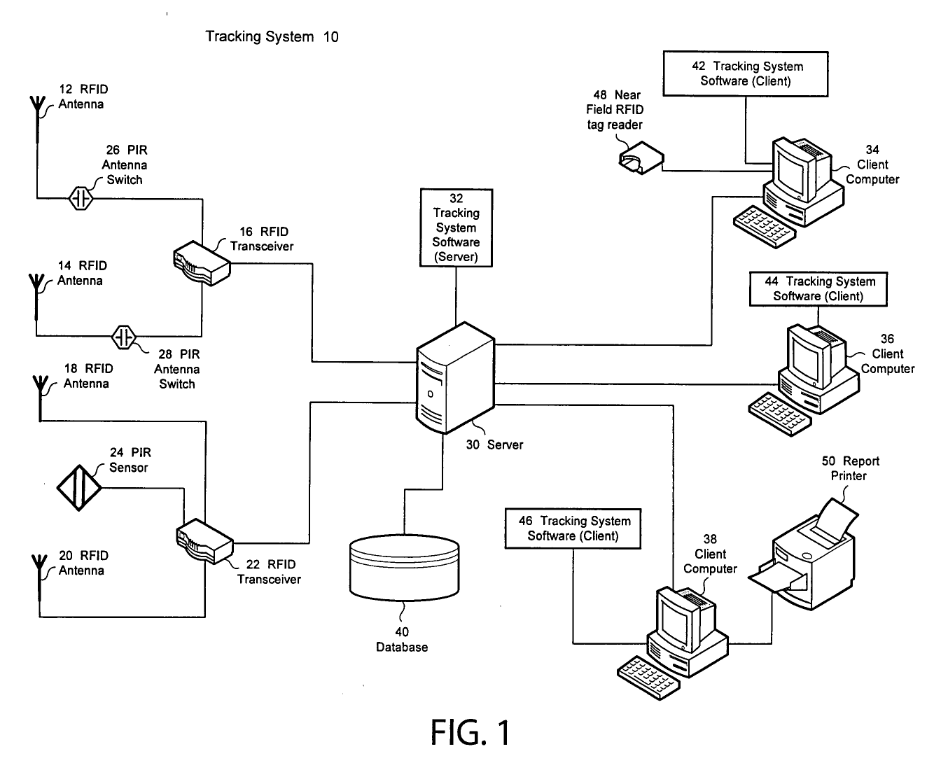

Referring now to the drawings where the illustrations are for the purpose of describing the preferred embodiment of the present invention and are not intended to limit the invention described herein, FIG. 1 is a schematic diagram of the fundamental components comprising the preferred embodiment of the tracking system 10 of the present invention. As illustrated in this Figure, RFID antennas 12 and 14 are connected to RFID transceiver 16, and RFID antennas 18 and 20 are connected to RFID transceiver 22. Each RFID transceiver 16, 22 is capable of independently controlling multiple RFID antennas. The antennas associated with any given RFID transceiver may be located in the same or different areas or zones of the tracking system 10, depending on the requirements of the specific installation. An optional PIR (passive infrared) presence sensor 24 is shown connected to RFID transceiver 22. RFID transceivers usually have inputs for PIR sensors and can be configured to enable their associated antennas only when a person or other infrared emitting object is present within the field of view and detecting range, hereinafter collectively referred to as field of view, of the PIR sensor. As illustrated, RFID antennas 12 and 14 are shown connected to RFID transceiver 16 through optional PIR (passive infrared) antenna switches 26, 28, respectively, which can be used when individual RFID antennas are connected to a common RFID transceiver and must be separately controlled. Such PIR antenna switches 26, 28 incorporate a PIR sensor, similar to PIR presence sensor 24, but the PIR sensor controls a switch that connects its associated RFID antenna to its associated RFID transceiver only when a person or other infrared emitting object is present within the field of view of the PIR sensor. When no such person or object is within the field of view of the PIR sensor, the PIR antenna switch disconnects the associated RFID antenna from its associated RFID transceiver and connects a non-radiating load to the RFID transceiver to prevent damage to the electronics contained within the RFID transceiver. The state of these switches is made available to the rest of the system via general purpose input/output (GPIO) connections that are commonly available on RFID transceivers.

The RFID transceivers 16, 22 are connected to a server 30 via a network, typically a LAN (local area network). The server 30 executes tracking system software 32 of the present invention. The software 32 incorporates logic that provides a determination of areas or zones in which RFID tags that have been successfully interrogated are present, regardless of whether the RFID tags can presently or continuously be successfully interrogated. Additionally, the software 32 of the present invention allows system administrators to configure the system 10, usually via client computers 34, 36 and 38. System configuration includes associating RFID antennas with predetermined areas or zones of the system, associating RFID tags with persons and/or objects of interest, and setting RFID operating parameters including RFID tag interrogation frequency, power radiation from the RFID antennas, and the like. The software 32 also utilizes a database 40 to store information for later use in reports and inquiries made by system users. System users typically access the system via the client computers 34, 36 and 38 connected to the server 30 via a network, typically a LAN. The client computers 34, 36 and 38 execute tracking system software 42, 44 and 46, respectively located thereon, allowing the computers 34, 36 and 38 to access the database 40, create reports, and access other features provided by the software 32 located on the server 30. The client computers 34, 36 and 38 may have near field RFID tag readers 48 attached thereto for use in initially recording RFID tag identifications and subsequently associating the tag identifications with persons and/or objects to be tracked by the system. Near field RFID tag readers are small desk top units capable of interrogating RFID tags only when the RFID tag is in close proximity to the reader, thus allowing the user to obtain the identifier from a specific RFID tag even though other RFID tags may be in the same room or lying on the same desk. The client computers 34, 36 and 38 may also have peripheral equipment, such as report printers 50, attached thereto for printing reports and otherwise distributing information provided by the system.

In order to implement the system of the present invention, an area or facility to be monitored is divided into zones of interest that do not overlap and comprise the entire area or facility to be monitored. In general, a zone of interest comprises a single functional area within a facility. Existing boundaries, such as rooms, groups of rooms, or even buildings are typically used to define the zones, however, an open area without walls can be divided into zones and monitored in the same manner. The zones of the system are defined and associated with a unique identifier within the software of the present invention when the system is deployed.

Each RFID antenna of the system is associated with, at most, one of the aforementioned zones, and each monitored zone is associated with at least one antenna. The number and position of RFID antennas associated with any zone is such that all persons and/or objects of interest must pass within the field of view and RFID tag interrogation range, hereinafter collectively referred to as field of view, of at least one antenna associated with the particular zone in order to enter that zone. The foregoing is accomplished by utilizing readily available commercial antennas of the appropriate design, physical positioning of the antennas, and carefully configuring the operating parameters of the antennas relative to the physical operating environment and the specific RFID tag being used. Different RFID tag types, such as passive and battery assisted passive (BAP), may require different antenna placement and different adjustments of antenna operating parameters. Different RFID tag designs of the same type may also require different tuning of the system to produce the desired field(s) of view. In an ideal installation, the fields of view of the antennas associated with different zones do not overlap. In many installations this is not possible, particularly in small areas near passageways through which persons and/or objects of interest may pass between zones. For example, an area immediately adjacent to a doorway connecting two different zones may be within the field of view of antennas associated with both zones. The present invention utilizes logic, hereinafter described, that provides an accurate and stable location determination of persons and/or objects of interest within these zones.

The RFID antennas utilized by the system of the present invention are connected to, and controlled and monitored by, a computer executing novel software. Associations between antennas and zones are entered into the system when the system is deployed. It should be noted that zones of the system can contain large areas that are not within the field of view of any antenna which can greatly reduce the cost of the system by reducing hardware and installation requirements, in addition to reducing disruptions in the operation of the facility caused by installation activities. For instance, when it is desired to determine whether a person or object of interest is outside the monitored area or facility, the area outside the monitored area or facility is treated like any other area by the system. In this case, at least one antenna associated with the area outside the facility must be positioned such that persons and/or objects of interest must pass through the field of view of at least one such antenna in order to leave the monitored area or facility.

Persons and/or objects of interest that are to be monitored by the system of the present invention are fitted with RFID tags that are capable of being interrogated by the antennas of the system. Each RFID tag contains a unique identifier that, along with the name of the associated person or object, is entered into the system of the present invention by the system user. When interrogated by an antenna of the system, the RFID tag transmits its unique identifier back to the activating antenna. Because the system of the present invention can associate any unique RFID tag identifier in its database with a particular person or object, collect the unique identifier of any RFID tag upon entry to any given zone by means of at least one antenna of the system, and associate any specific antenna with a particular zone, the software utilized by the system of the present invention can logically determine that any person or object detected within a specific zone continues to be present within that zone, regardless of whether that person or object is detected by any other antenna of the system until the person or object is detected in a different zone.

A given area or facility may also contain zones that are not monitored, i.e., the zone is not associated with any given RFID antenna of the system. If passageways exist between such an unmonitored zone and an adjacent monitored zone, and the monitored zone is not completely within the field of view of antenna(s) associated with the monitored zone, a person and/or object of interest that enters and then subsequently exits the field of view of antenna(s) associated with the monitored zone is determined by the system of the present invention to be located either in the monitored zone or the unmonitored zone. This determination will continue to be made by the system until the person and/or object of interest enters the field of view of any antenna that is associated with any monitored zone of the system. The presence of unmonitored zones does not present a problem for some system users and, if this is the case, such unmonitored zones can provide additional cost savings. For example, consider a correctional facility in which a monitored zone, such as a housing unit, is connected to an unmonitored zone, such as a hallway. As is typical in such cases, the hallway is only visually monitored by a control room rather than by the system of the present invention, doorways connecting the monitored housing unit to the hallway are kept locked, and inmates enter the hallway only when escorted by a guard who must also open the locked door. In such a case it may be sufficient to be aware that the inmate is either in the housing unit or in the adjacent hallway with an escort.

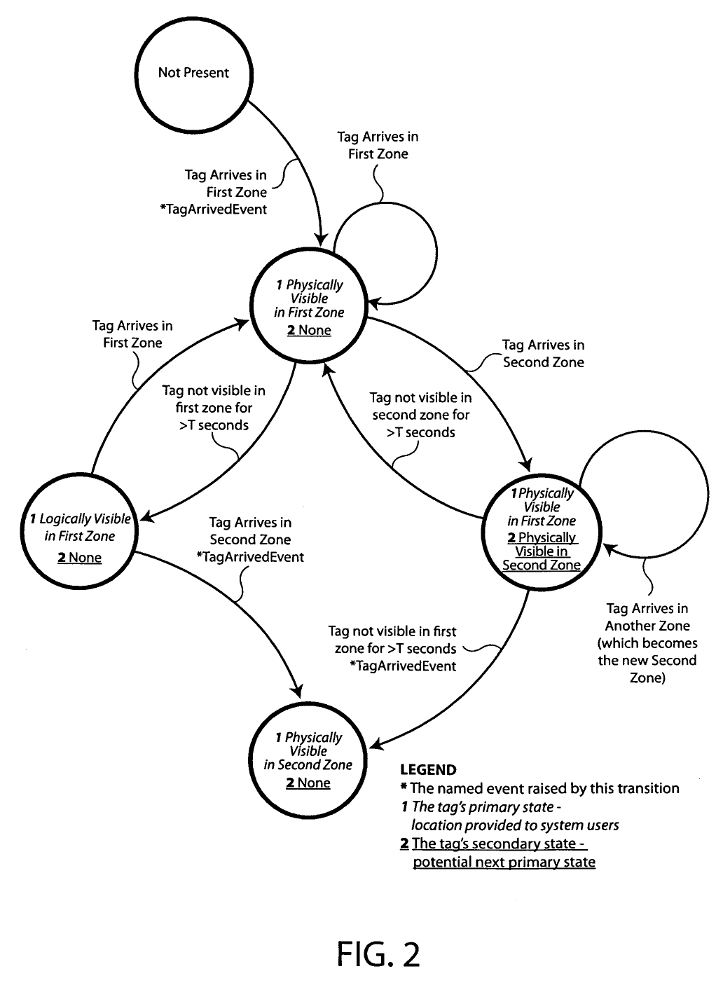

As previously discussed, there may be small areas adjacent to doorways or other passageways connecting two zones that are within the fields of view of antennas in both zones. In this case, a person or object fitted with an RFID tag and passing such a doorway but not changing zones would be temporarily detected by an RFID antenna associated with the adjoining zone. In this and similar situations the receiver signal strength indication (RSSI) from the two spatially separated antennas may be very similar, making RSSI an unsuitable means by which to determine whether the RFID tag and, by association, the person or object of interest has entered a different zone. In some physical configurations it is even possible that the RSSI for an antenna associated with an adjoining zone may be temporarily larger than that for the zone occupied by the RFID tag in question. Without additional compensating logic such situations could result in rapidly fluctuating and/or inaccurate determinations as to the location of a given RFID tag and the associated person and/or object of interest. In order to provide more stable and accurate location determinations, the software utilized by the system of the present invention incorporates logic illustrated in FIG. 2 which shows the possible states of an RFID tag and how such states are determined by the software utilized by the system of the present invention.

Referring to FIG. 2, when a specific RFID tag becomes physically visible to an antenna associated with a first zone and subsequently becomes simultaneously visible to an antenna associated with a second zone, the system of the present invention continues to determine that the RFD tag is located in the first zone until it becomes physically invisible to every antenna associated with the first zone for longer than a predetermined time interval, while remaining visible to any antenna associated with the second zone. At this time the location of the RFID tag is determined to be located in the second zone, which is the only zone associated with an antenna to which the RFID tag is visible. This occurs when an RFID tag moves from a first zone into a second zone through an area where the fields of view of antenna associated with both zones overlap. It should be noted that when an RFID tag in a first zone is within the field of view of an antenna associated with the first zone and then temporarily passes through an area that is also within the field of view of an antenna associated with a second zone, the system of the present invention properly determines that the RFID tag has remained in the first zone. If the RFID tag had actually moved into a second zone it would eventually become detected in the second zone while becoming invisible in the first zone, at which point the system would properly determine that the RFID tag is located in the second zone.

In cases where a specific RFID tag becomes physically visible to an antenna associated with a first zone and subsequently becomes physically invisible to any antenna associated with the first zone for longer than a predetermined time interval, while not becoming physically visible to any antenna associated with another zone of the system, the system determines that the RFID tag is logically visible in the first zone, and also determines that it is in the first zone. If the RFID tag subsequently becomes physically visible to an antenna associated with a second zone, the system determines that the RFID tag is located in the second zone, which is the only zone associated with an antenna to which the RFID tag is visible. This occurs when an RFID tag moves from a first zone into a second zone through an area not covered by the field of view of any antenna. It should be noted that when an RFID tag in a first zone is within the field of view of an antenna associated with the first zone and passes into an area not within the field of view of any antenna, the system properly determines that the RFID tag has remained in the first zone. If the RFID tag had actually moved into a second zone it would eventually become detected in only the second zone, at which time the system would determine that the RFID tag is located in the second zone. It should be further noted that it is possible to devise an RFID tag state diagram and associated program logic that takes into account areas where the fields of view of antennas associated with more than two different zones overlap, however, the proper set-up of system antennas and their associated fields of view, in accordance with the previously discussed requirements, makes this unnecessary.

In addition to determining in which zone a person or object of interest is currently present, the present invention records a tag arrived event each time an RFID tag is determined by the software of the present invention to have moved to a different zone. These events are used by the system to update the current location of the RFID tag and for historical reference. For example, this information can be used to show when a particular person of interest visited a specific zone and it can provide a historical sequence of zones visited by a person of interest within a given period of time. It should be noted that the time at which an RFID tag exits a specific zone is the time at which it enters any different zone, so the period of time spent in any monitored zone of the system can be determined.

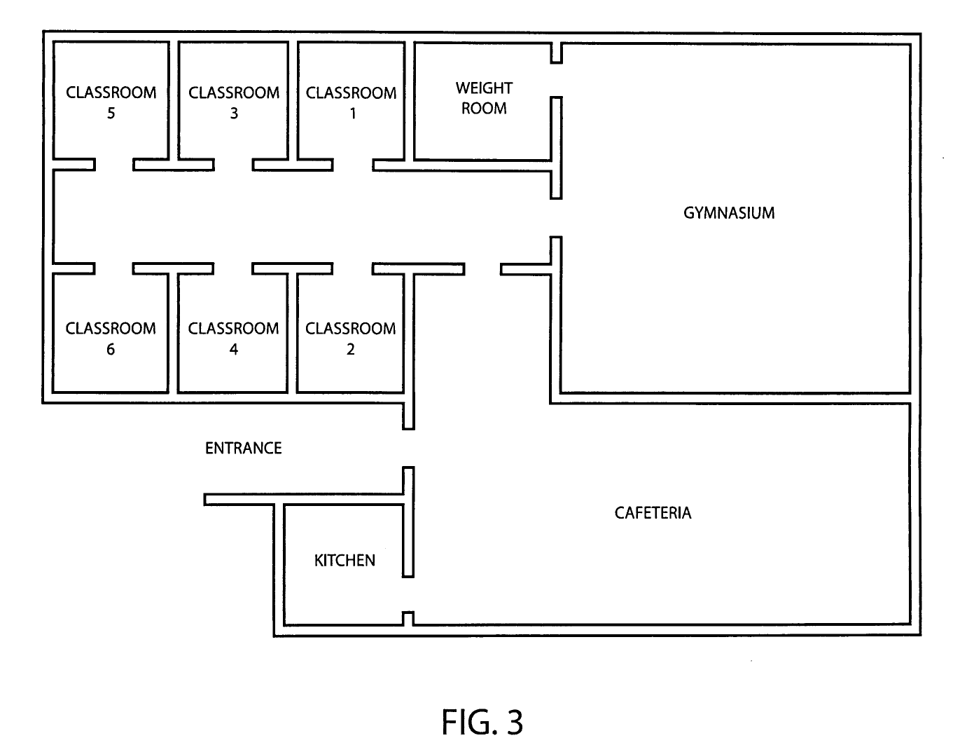

Referring now to FIG. 3, an installation of the preferred embodiment of the present invention in a simplified setting is illustrated. In this case the simplified setting comprises a facility consisting of six classrooms (educational area), a gymnasium with a weight room (recreational area) and a cafeteria and kitchen (food service area). The configuration of the system begins by determining how the facility is best divided into zones, taking into account the needs of the system users. It is assumed that the system users need only to determine whether persons of interest are in the facility and, if so, whether they are in the educational, recreational, or food service areas. Given the floor plan and the needs of the system user, the goals of the user can be readily accomplished with only four zones consisting of:

Zone 1—Educational area (6 classrooms and connecting hallway)

Zone 2—Recreational area (gymnasium and weight room)

Zone 3—Food service area (cafeteria and kitchen)

Zone 4—Everything outside the facility

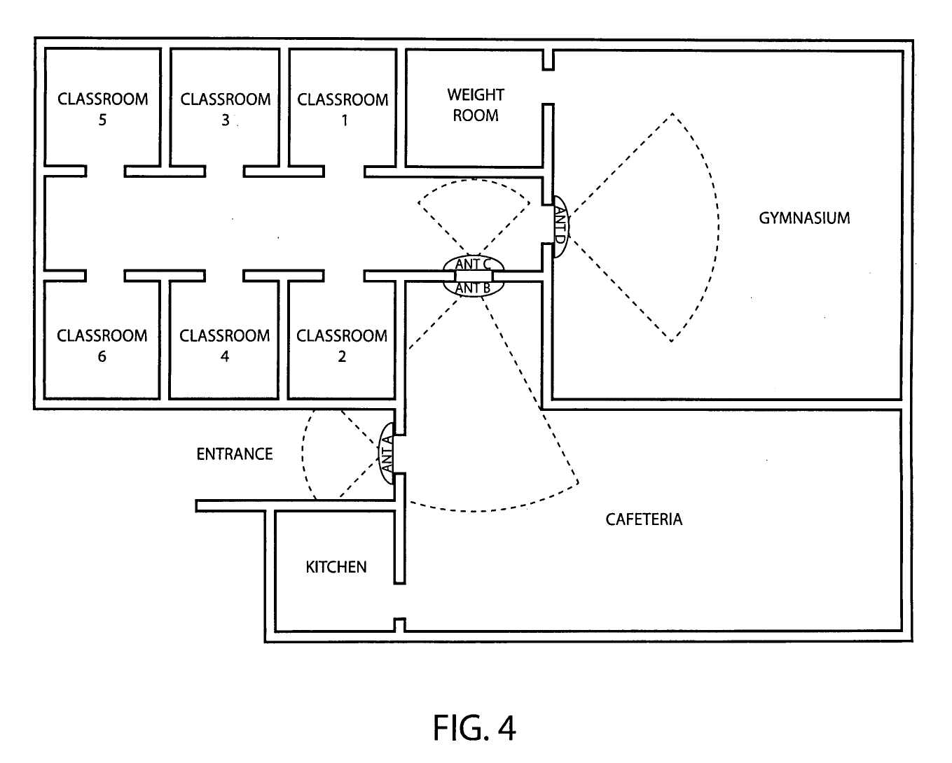

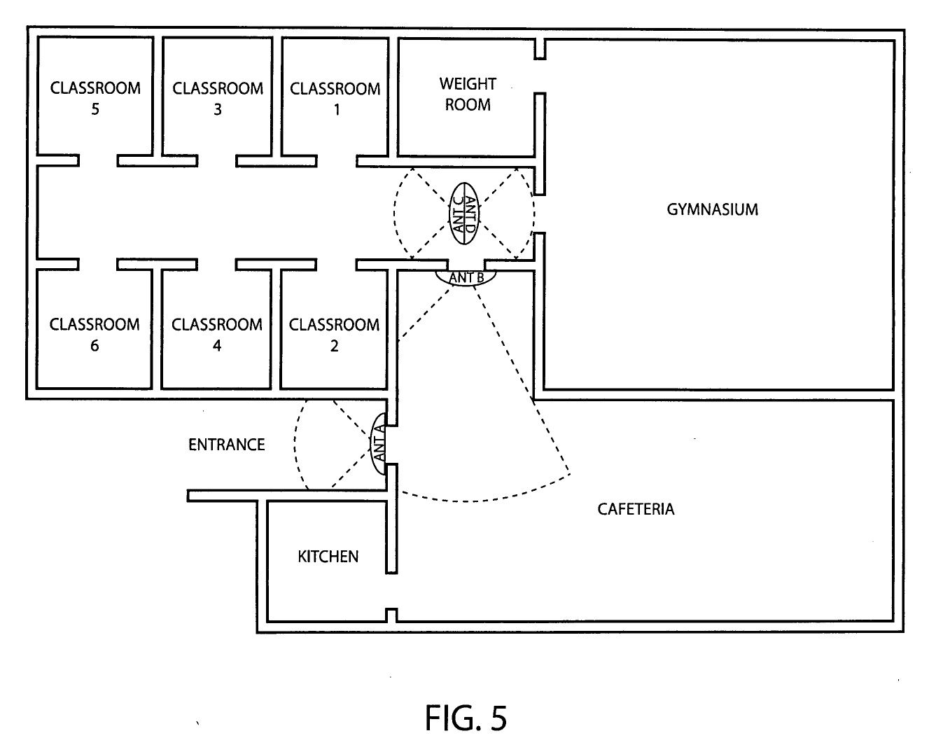

In this instance only four antennas positioned as shown in FIG. 4, are required. It should be noted that positioning antennas “ANT C” and “ANT D” in a back-to-back relationship, as shown in FIG. 5, is advantageous because positioning as many of the required antennas as possible in close proximity to one another minimizes cable length, overall system installation cost, and minimizes any disruptive activity associated with the installation of same in the facility. The foregoing antenna arrangement requires including a small portion of the hallway within the recreational area zone, which would typically be of no consequence. Given the antenna placement in either FIG. 4 or 5, the following table lists the antenna which last successfully interrogates (detects) a specific RFID tag versus the present location of the person or object of interest associated with the RFID tag.

| Antenna that last successfully | Present location of |
|---|---|
| interrogated a given RFID tag | associated person/object |
| Antenna “ANT A” | Locations outside the facility |
| Antenna “ANT B” | Food Service Area |
| Antenna “ANT C” | Educational Area |
| Antenna “ANT D” | Recreational Area |

In some circumstances tracking systems need only determine whether a person or object of interest has visited a certain location and not determine the present location of the person or object. In such a case, antenna “ANT A” in FIGS. 4 and 5 can be eliminated resulting in a lower cost while accomplishing that which is required of the system. It should be noted that without antenna “ANT A”, it is possible to determine whether a specific person or object of interest visited a particular zone and when the visit occurred, but it is impossible to determine with certainty the present location of a person or object of interest if the antenna that last successfully interrogated the associated RFID tag is antenna “ANT B”. In this case the present location of the person or object of interest is either the food service area or is outside the facility.

Optionally, the present invention can incorporate passive infrared (PIR) sensors at or near some or all of its RFID antennas, as shown in FIG. 1. The PIR sensors enable the interrogation of the RFID tags by the associated RFID antenna(s) and/or RFID transceiver(s) only when an infrared emitting source, such as a person, is within the field of view of the PIR sensor. When an infrared emitting source is not within the field of view of the PIR sensor, the interrogation of the RFID tags by the associated RFID antenna(s) and/or RFID transceiver(s) is disabled. The field of view of the PIR sensor is adjusted physically and/or electronically to be substantially within the field of view of its associated RFID antenna(s). Incorporating PIR sensors in this manner prevents RFID tags from being activated in fringe areas of the antenna's field of view, where repeated interrogation attempts may be received by the RFID tag but the response signal of the RFID tag may not be strong enough to be detected by the antenna and/or its associated RFID transceiver. This can help extend battery life in battery assisted RFID tags by reducing the amount of time during which any given RFID tag is interrogated. In addition to conserving power and extending battery life, in some situations using PIR sensors in this manner can also help avoid the detection of RFID tags in adjacent zones. Additionally, this implementation of PIR sensors allows the previously described system to detect persons who are not wearing RFID tags or are wearing non-functional RFID tags. If a PIR sensor of the system is activated but there is no corresponding RFID tag detected by the associated RFID antenna, logic within the system software determines that a person within the monitored area is not wearing a RFID tag, or is wearing a broken or defective RFID tag, and corresponding alerts and/or reports can then be produced by the system. Other types of presence sensors, such as pressure mats, sound detectors, and beam break sensors can be similarly employed to either augment or replace the PIR sensors.

An alternate embodiment of the present invention incorporates PIR sensors as previously described, except that the PIR sensors are not used to enable or disable their associated RFID antenna(s). Instead the PIR sensors provide only an indication as to their status (person detected or person not detected) to the software of the present invention. Because the RFID antenna(s) in this embodiment are always enabled, if a PIR sensor is activated but there is no corresponding RFID tag detected by the associated antenna, logic within the system software determines that a person within the monitored area is not wearing an RFID tag, or is wearing a broken or defective RFID tag. This implementation of PIR sensors does not provide the power saving and other benefits of the PIR sensor implementation described in the preferred embodiment.

Another alternate embodiment of the present invention involves an adaptation of the preferred embodiment utilizing zones that are disjointed and do not share any boundaries with other zones. These zones may be separated by significant distances. In this configuration the system can determine if and when, a person or object of interest fitted with an RFID tag visited a specific area. No other determination with respect to the location of the person or object can be made unless the zone(s) are completely covered by the field of view of their respective antennas, in which case the duration of the visit can be determined.

One application of this alternate embodiment of the present invention is monitoring whether a person fitted with an RFID tag has reported to a specific location at the correct time. An example of this is a security officer's tour where the security officer is fitted with an RFID tag. Designated zones are established at locations within the facility that the security officer must visit when making his or her rounds and the system can determine when the officer visited these locations. Additional software logic allows the system to determine whether the officer's visits to these locations were made at the proper predetermined dates, times, and/or frequencies, and based on these determinations, the system can issue reports and/or alarms.

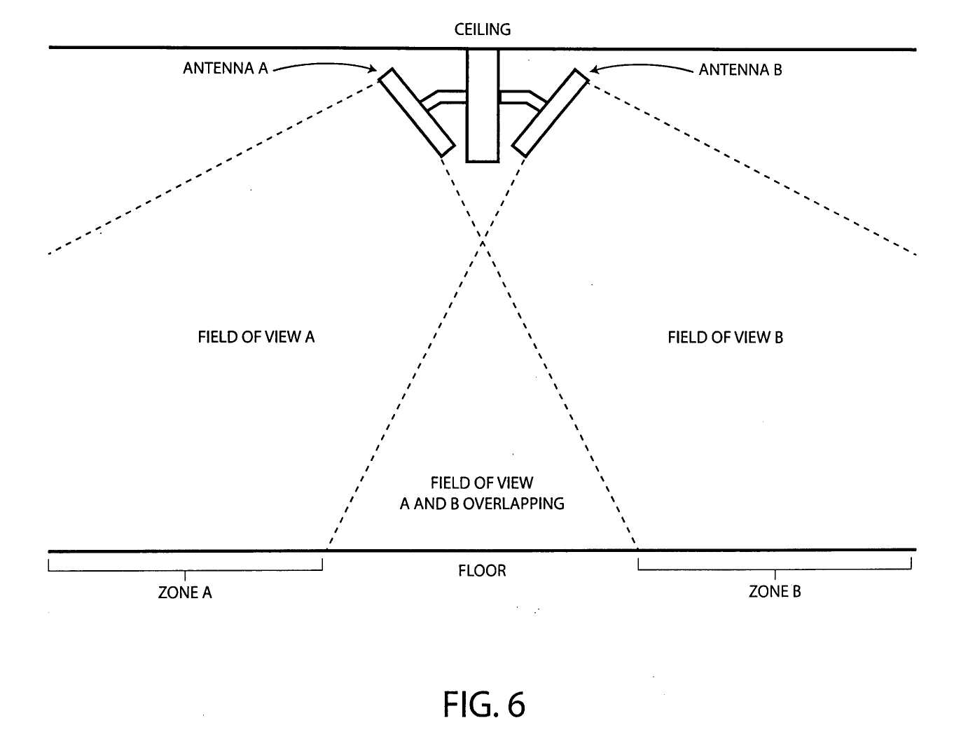

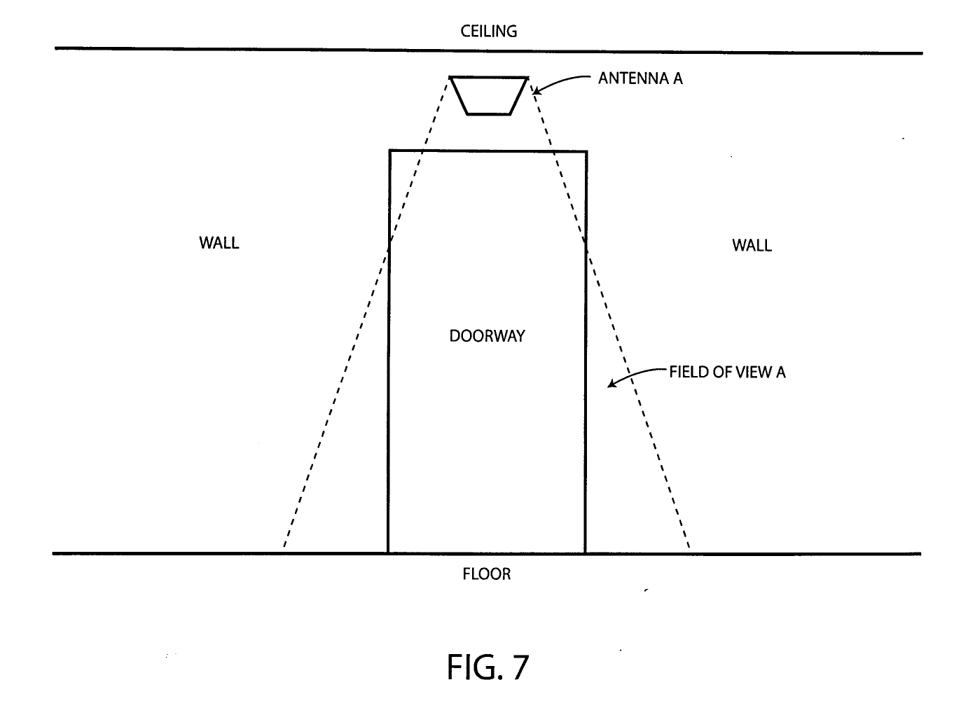

Still another alternate embodiment of the present invention involves an adaptation of the preferred embodiment of the present invention in which all passageways between adjacent defined zones are provided with a pair of antennas, A and B, which are respectively associated with each of the two adjacent zones of the system. These antennas are typically positioned in a back-to-back relationship on opposite sides of a wall containing a doorway or other passageway through the boundary of the specific zone, as shown in FIGS. 6 and 7. The center of the field of view of antenna A of each pair is set to extend toward its associated zone and downward at a steep angle, and adjusted to create a relatively small field of view encompassing a span through which persons or objects of interest bearing RFID tags must pass in order to enter the zone. The center of the field of view of antenna B is similarly adjusted relative to its associated zone. The fields of view of these antennas should not overlap, but if overlap is unavoidable, it should be minimized, as shown in FIG. 6. Adjusting the shape, position, and size of fields of view of the antennas is accomplished by selecting readily available commercial antennas of appropriate design, tuning the operating parameters of the antennas, physically positioning the antennas, and using radio frequency shielding materials, if necessary.

In this alternate embodiment of the present invention, the antennas are set to attempt RFID tag interrogation at a high rate, e.g., several hundred times per second. When a person or object bearing an RFID tag moves from zone A to zone B, the RFID tag will first be detected only by antenna A, then by both antennas A and B if their fields of view overlap, and then only by antenna B. Due to the high tag interrogation rate, a large number of successful RFID tag interrogations will occur on only antenna A, followed by a few or no successful RFID tag interrogations on both antennas A and B depending on whether and the degree to which their respective fields of view overlap, followed by a few successful RFID interrogations on only antenna B. It should be noted that only a few successful RFID interrogations of the RFID tag occurs from antenna B because the human body is opaque to the RFID signals and “shadows” the RFID tag when the person is moving away from the antenna, assuming that the RFID tag is worn on the front of the person's body. This situation can be remedied with the use of additional antennas in other orientations, and/or RFID tag inlays of a different design, and/or adjusting antenna positions, but since only one successful RFID tag interrogation from antenna B is all that is required, these remedies are seldom necessary. In any event, an RFID tag that is first successfully interrogated only by antenna A and subsequently successfully interrogated only by antenna B, whether or not there are any intervening successful RFID tag interrogations from both antennas, will be deemed by the system to have passed from zone A to zone B.

The system software is programmed to determine the occurrence of the aforementioned pattern or sequence of successful RFID tag interrogations from the paired antennas positioned as previously described. Thus, it is possible for the software logic to determine:
1) The identity of a person or object of interest between any two zones of the system.
2) The zone that the person or object of interest exited.
3) The zone that the person or object of interest entered.
4) The specific passageway used by the person or object of interest between the two zones.
   Furthermore, even if the RFID tag is no longer within the field of view of any antenna, the system software will continue to determine that the person or object of interest is present in the same zone until that person or object of interest is detected passing into a different zone through any similarly monitored passageway. It should be noted that the same antenna configuration can be used regardless of whether the given zone is adjacent to another zone of the system.

In the case where the zones of the system are disjointed and monitored passageways lead to an unmonitored area, the antenna placement is as previously described with one antenna of the pair of antennas having its field of view oriented toward the unmonitored area. In this case, the unmonitored area is regarded as another zone of the system, and the system can still determine within which zone a person or object fitted with an RFID tag is present. This arrangement is suitable for monitoring entry and exit from buildings when unmonitored areas between zones are accessible only with an escort or when these areas are secured by other means.

A further embodiment of the present invention is an adaptation of the preferred embodiment in which a camera-based system utilizes facial recognition technology in place of RFID tags and associated antennas. Other components of the system are as previously described.

Because such a system can
1) Associate any face known to the system with a particular person of interest,
2) Collect facial recognition data (a picture) upon entry to any given zone by means of at least one camera, and
3) Associate any given camera with a specific zone,
   the system software can logically determine that any person detected within a given zone continues to be present within that zone, regardless of whether that person or object is presently detected by any system camera, until the person or object is similarly detected in a different zone.

A still further embodiment of the present invention is an adaptation of the preferred embodiment in which RFID tags are replaced by a unique optically recognizable pattern (such as a two-dimensional bar code) placed upon persons and/or objects to be tracked, and cameras or other optical sensors (scanners) are utilized in place of RFID antennas. Since uniforms are provided to persons in some environments, it is possible to present an optically recognizable pattern on several areas of the uniform such that it can be recognized by the optical scanners regardless of the person's orientation with respect to the sensors.

Certain modifications and improvements will occur to those skilled in the art upon reading the foregoing. It is understood that all such modifications and improvement have not been included herein for the sake of conciseness and readability, but are properly within the scope of the following claims.

We claim:

1. A method of determining which subarea within a plurality of subareas comprising an area to be monitored contains an object of interest that may move between said subareas comprising the steps of:
a) associating said subareas with at least one detecting means, each said detecting means being associated with only one subarea, said detecting means being capable of detecting the presence of said object of interest within at least a portion of said subarea through which said object of interest must pass when within said subarea;
b) creating a record that said object of interest was detected by said detecting means after being undetected by said detecting means for a predetermined period of time; and
c) analyzing said record according to predetermined rules to determine the specific subarea in which said object of interest is present.

2. The method as defined in claim 1 wherein said object of interest is fitted with a unique identifier and said detecting means is capable of reading said unique identifier and associating said unique identifier with said object of interest.

3. The method as defined in claim 2 wherein said unique identifier is contained within a transponder device which transmits said unique identifier when activated and said detecting means is capable of activating said transponder device and receiving said unique identifier transmitted by said transponder device.

4. The method as defined in claim 3 wherein said transponder device is an RFID tag and said detecting means is comprised of an RFID antenna and an associated RFID transceiver.

5. The method as defined in claim 2 wherein said detecting means further includes a computer device that performs said association of said unique identifier with said object of interest and analyzes said record according to said predetermined rules.

6. The method as defined in claim 1 wherein said record includes at least the identity of said object of interest, the time at which said object of interest was detected, and the subarea associated with said detecting means which detected said object of interest.

7. The method as defined in claim 1 wherein said subareas within said plurality of subareas are contiguous.

8. The method as defined in claim 1 wherein said subarea within said plurality of subareas is contiguous to an unmonitored subarea.

9. The method as defined in claim 1 wherein said predetermined rules are based upon known movement patterns of said object of interest in said subareas within said area.

10. The method as defined in claim 1 wherein said object of interest is a person and said detecting means comprises camera equipment operatively attached to a computer device capable of recognizing features of said object of interest.

11. A system for determining the location of an object of interest within an area to be monitored comprising at least one antenna having a field of view within a subarea of a plurality of subareas comprising said area to be monitored, a transceiver device associated with said at least one antenna, a transponder device operatively attached to said object of interest, said transponder device containing a unique identifier for said object of interest, said at least one antenna activating said transponder device when said object of interest is within said field of view of said at least one antenna causing said transponder device to transmit said unique identifier to said at least one antenna, means for associating said unique identifier with said object of interest, and means for creating a record that said unique identifier was detected by said transceiver device indicating that said object of interest was within said subarea after being undetected in said subarea for a predetermined period of time.

12. The system as defined in claim 11 wherein the maximum field of view of said at least one antenna substantially approximates the size of said subarea of said plurality of subareas comprising said area to be monitored.

13. The system as defined in claim 11 wherein the maximum field of view of said at least one antenna is substantially smaller than the size of said subarea of said plurality of subareas comprising said area to be monitored.

14. The system as defined in claim 11 wherein said transponder device is an RFID tag and said at least one antenna is an RFID antenna and its said associated transceiver device is an RFID transceiver.

15. The system as defined in claim 11 wherein said unique identifier associating means comprises a computer device that performs said association of said unique identifier with said object of interest.

16. The system as defined in claim 15 wherein said computer device establishes the operating parameters of said transceiver device and its associated at least one antenna.

17. The system as defined in claim 15 wherein said computer device associates said at least one antenna with said subarea within said plurality of subareas comprising said area to be monitored.

18. The system as defined in claim 15 wherein said computer device receives and stores data from said transceiver device.

19. The system as defined in claim 18 wherein said computer device analyzes said data according to predetermined rules to determine the specific subarea in which said object of interest is present.

20. The system as defined in claim 11 further including a presence sensing device capable of:
a) detecting the presence of said object of interest only when said object of interest is located substantially within said field of view of said at least one antenna;
b) enabling the activation of said transponder device by said at least one antenna and/or said transceiver device only when said object of interest is detected by said presence sensing device; and
c) disabling the activation of said transponder device by said at least one antenna and said transceiver device unless said object of interest is detected by said presence detecting device.

21. The system as defined in claim 11 further including at least one remote computer device, said at least one remote computer device being operatively connected to said computer server device permitting said at least one remote computer device to act in conjunction with said computer device to establish said operating parameters of said transceiver device and its associated at least one antenna.

22. The system as defined in claim 21 further including peripheral equipment operatively connected to said at least one remote computer device.

23. The system as defined in claim 22 wherein said peripheral equipment comprises printer devices.

24. The system defined in claim 21 further including a transceiver device operatively connected to said at least one remote computer device.

25. The system as defined in claim 11 wherein said at least one antenna comprises two antennas arranged in a back-to-back relationship with their respective fields of view being substantially contained within different subareas.
